(19) United States
(12) Patent Application Publication
(10) Pub. No.: US 2017/0196511 A1
(43) Pub. Date: Jul. 13, 2017

(54) ELECTROPHYSIOLOGICAL TESTING DEVICE

(71) Applicant: MOBILEMEDTEK HOLDINGS, INC., Louisville, KY (US)

(72) Inventors: Bryan EHRET, Crestwood, KY (US); David KENNEDY, Georgetown, IN (US); Steve UPCHURCH, Louisville, KY (US); Sumner E.P. ADAMS, NE Corydon, IN (US)

(21) Appl. No.: 15/304,946

(22) PCT Filed: Apr. 20, 2015
(86) PCT No.: PCT/US15/26605

§ 371 Date: Oct. 18, 2016

Related U.S. Application Data
(60) Provisional application No. 61/981,488, filed on Apr. 18, 2014.

(51) Int. Cl. A61B 5/00; A61B 5/0492; A61B 5/0478

(52) U.S. Cl. CPC A61B 5/6803; A61B 5/725; A61B 5/7225; A61B 5/0478; A61B 5/0006; A61B 5/0492; A61B 2560/0431; A61B 5/0531

(57) ABSTRACT

A portable device for use in performing a plurality of different electro-physiological tests is provided. The device may include a port for receiving and a controller for processing signals from each of the different tests. The device may also receive a portable computing device such as a laptop or tablet for use in analyzing the signals processed by the device. A headgear including straps may be used to perform one or more of the tests. The headgear may include one or more connectors for connecting the straps of the headgear, for connecting electrodes to a patient, and for maintaining contact between the electrodes and the patient's head during the test. In addition, the device may be capable of assisting a user of the device in configuring the electrodes for the different tests.

This application claims priority to U.S. Provisional Patent Application No. 61/981,488, filed Apr. 18, 2014, the disclosure of which is incorporated herein by reference.

### FIELD OF INVENTION

This invention is directed to electrophysiological testing and, more particularly, to a device that facilitates physical and electrical connection of one or more electrophysiological testing wires.

### BACKGROUND OF THE INVENTION

A multitude of electrophysiological tests are currently available for medical professionals to perform on subjects in order to analyze the electrical processes of the body, including, but not limited to, electroencephalography (EEG), electromyography (EMG), nerve conduction studies (NCS), evoked potentials (EP), transcutaneous electrical nerve stimulation (TENS), Holter monitoring, and electrocardiography (ECG/EKG). Such electrophysiological tests are currently performed via a multitude of expensive, bulky, time consuming devices with limited mobility. This time consumption and limited mobility hinders the medical professional's ability to perform such electrophysiological tests quickly, or in a multitude of locations and on a variety of subjects, including, but not limited to, non-ambulatory subjects. Medical professionals must either own or otherwise have access to a variety of testing devices, all of which must be maintained. In addition, there are a variety of tests which could provide more comprehensive results if performed over a length of time or at a location that is infeasible for the medical professional to accommodate due to limited availability of exam room space or the physical presence of the medical professional. For example, a subject who may be experiencing intermittent seizure activity may not manifest said seizure activity during a standard test performed in the office of a medical professional, and a lengthy, portable, recorded test would offer higher testing yield. An example for the need of expedited test performance would be a critical subject with a head injury that may be suffering from prolonged seizures. The current expensive, bulky equipment is unable to be stored in areas that are easily accessible in critical care scenarios. A smaller, more portable unit with time saving setup procedures and on demand viewing potential would allow for expedited test setup and performance times, which could dramatically improve the quality of care in a critical care scenario.

A system must be provided which will allow a medical professional to perform electrophysiological testing faster and in a variety of locations without the inconvenience or risk involved with transporting expensive, bulky devices or without extensive and timely setup procedures. This system should allow the medical professional to perform a multitude of electrophysiological tests with one small, portable, time efficient, affordable device. This system should also make the results of the electrophysiological tests available to medical professional in a multitude of locations and in real time.

### SUMMARY OF THE INVENTION

One object of the present disclosure is to provide a portable apparatus for receiving and processing electrophysiological signals from a patient related to a plurality of different electrophysiological tests.

One embodiment relates to an apparatus for use in association with a portable computing device in performing a plurality of different electrophysiological tests on a patient. The apparatus may include a body including a receiver for connecting to and receiving at least a portion of the portable computing device, and a controller for receiving a plurality of electrophysiological signals from the patient and for processing and transferring said electrophysiological signals to the portable computing device. The body may further include a central shell with at least one opening for allowing access to a keyboard or touch interface of the portable computing device when the portable computing device is received by and connected to said body. In addition, the apparatus may include means for accessing at least one tactile button on the portable computing device. For example, the means may comprise an aperture in the body for directly accessing the tactile button, or an actuatable portion of the body for actuating the tactile button.

In one aspect, the body may include a first portion on a first side of the central shell and a second portion on a second side of the central shell, wherein the first portion includes the controller. Each of the first and second portions may include a recessed grip adapted to allow a user to hold the body and the portable computing device.

In another aspect, the controller may be adapted to be powered by the portable computing device.

The apparatus may include a connector port adapted to receive the plurality of electrophysiological signals from the patient via a plurality of different configurations of electrodes and further adapted to transmit said signals to the controller for processing. The body may further include at least one port for direct electrical communication with an input port associated with the portable computing device.

Another object of the present disclosure is to provide an apparatus for use in connecting wires or other electrophysiological signal transmitters to a patient's head in specific locations for use in an electrophysiological test associated with the head.

One embodiment relates to an apparatus for use in locating and attaching electrophysiological signal transmitters to a head of a patient, said electrophysiological signal transmitters adapted to receive electrophysiological signals from the patient and transmit said signals to a system for processing said signals. The apparatus may include a plurality of bands for positioning on the head of the patient, said bands adapted for linear proportionate elongation, and a plurality of connectors for connecting the electrophysiological signal transmitters to the plurality of bands and for maintaining contact between the transmitters and the head of the patient.

In one aspect, at least one of the plurality of connectors is adapted to connect a first of the plurality of bands to a second of the plurality of bands. In another aspect, at least one of the plurality of bands includes markings thereon for measuring a distance from a predetermined point.

The plurality of bands may comprise a vertical band adapted for placement along a central portion of the head from front to back and a plurality of lateral bands adapted for placement over the head from side to side. The plurality of bands may further include a circumferential band adapted to substantially surround a circumference of the head. In addition, the apparatus may include a primary positioning element for attachment to and location of the plurality of bands, said primary positioning element including a nose support and Nasion location element. The primary positioning element may further include an Inion location element.

In one aspect, the vertical band and the plurality of lateral bands may form an integrated, unitary element.

In another aspect, the connectors may comprise a plurality of slots for slidably receiving at least one of the plurality of bands, and at least one receiver for receiving at least one of the electrophysiological signal transmitters. The connectors may further include at least two layers connected by a hinge, said hinge adapted for opening the at least two layers for placing at least one of the plurality of bands within at least one of the plurality of slots. In a further aspect, the connectors may include a controller for amplifying the electrophysiological signals and wirelessly transmitting said signals.

In another embodiment of the present invention, a method is disclosed for configuring one of a plurality of wiring harnesses attached to a patient, said wiring harnesses including a plurality of electrodes for use in conducting one of a plurality of different electrophysiological tests on the patient, each of said wiring harnesses including an internal code specific to one of the plurality of different tests. The method may include providing an electronic testing system adapted for connecting to each of the plurality of different wiring harnesses, said electronic testing system for receiving and processing electrophysiological signals from the patient. In addition, the method may include identifying a first of the wiring harnesses connected to the electronic testing system by a first internal code associated with the first wiring harness, and identifying a fist map of a plurality of electrode locations associated with conducting a first electrophysiological test with the first wiring harness, said first map accessible by the electronic testing system. Further, the method may include prompting a user to identify each of the plurality of electrodes of the first wiring harness corresponding to the electrode locations. The system may be adapted for receiving an input from the user identifying each of the plurality of electrodes of the first wiring harness, and mapping each of the plurality of electrodes of the first wiring harness to each of the electrode locations of the first map.

### BRIEF DESCRIPTION OF THE DRAWINGS

FIG. 1 is a perspective view of a portable device for performing a plurality of different electrophysiological tests according to the present invention;

FIG. 2 is a rear elevational view of the device of FIG. 1;

FIG. 3 is a wire for use with the device of FIG. 1;

FIG. 4 is a top plan view of a connector of the device of FIG. 1;

FIGS. 5a-5e illustrate various configurations of the device of FIG. 1;

FIG. 6 is a schematic of a controller of the device of FIG. 1;

FIG. 7 is a perspective view of a head apparatus of the present invention;

FIG. 8a is a primary alignment band of the head apparatus of FIG. 7;

FIG. 8b is a main horizontal band of the head apparatus of FIG. 7;

FIG. 8c is a central vertical band of the head apparatus of FIG. 7;

FIG. 8d is a central lateral band of the head apparatus of FIG. 7;

FIG. 8e illustrates front and rear lateral bands of the head apparatus of FIG. 7;

FIG. 9 is a central and lateral strap system according to one embodiment of the head apparatus of FIG. 7;

FIGS. 10a and 10b illustrate a connector for use with the head apparatus of FIG. 7; and

FIG. 11 is a schematic diagram of the steps of a method of configuring electrodes according to the present invention.

### DETAILED DESCRIPTION OF THE INVENTION

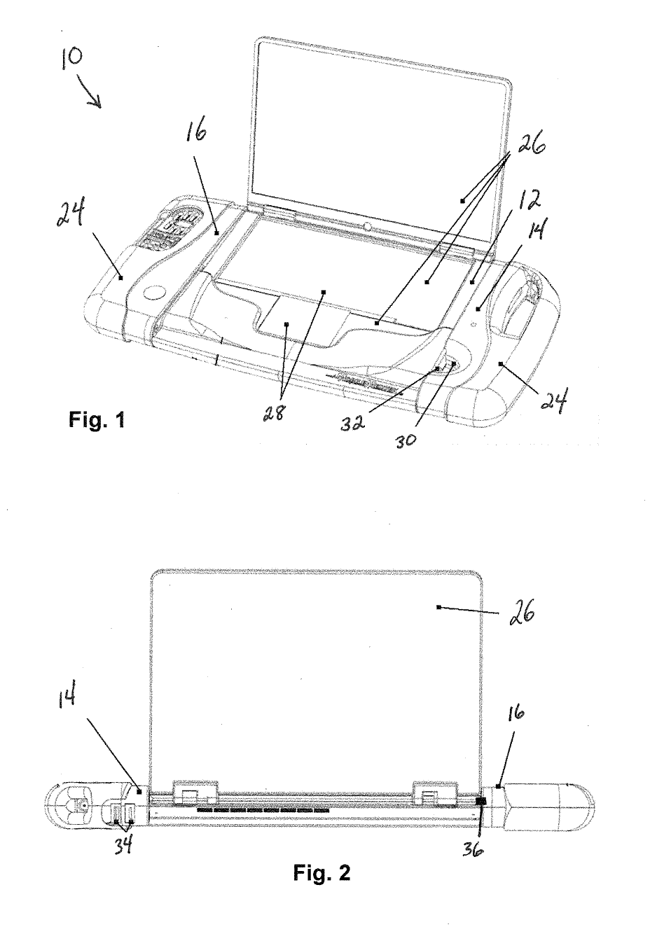

FIGS. 1-6 illustrate one embodiment of a portable device 10 that may be used in performing medical testing, such as is described in U.S. patent application Ser. No. 13/038,940, the disclosure of which is incorporated herein by reference.

In the illustrated embodiment, FIG. 1, the device 10 comprises a centralized shell 12 designed to house a mobile device such as a laptop or tablet computer. The device 10 also comprises a right outrigger 14, which houses the power element and USB/HDMI break outs for the mobile device 10, as well as a left outrigger 16, which houses the analog, digital and connector components that allow for the referenced medical testing. Both the right outrigger 14 and the left outrigger 16 may have recessed handles or grips 24 that allow for easy holding and manipulation of the device 10. The ordinarily skilled artisan will understand that the relative positions of each outrigger and the location of the contents thereof may be interchangeable.

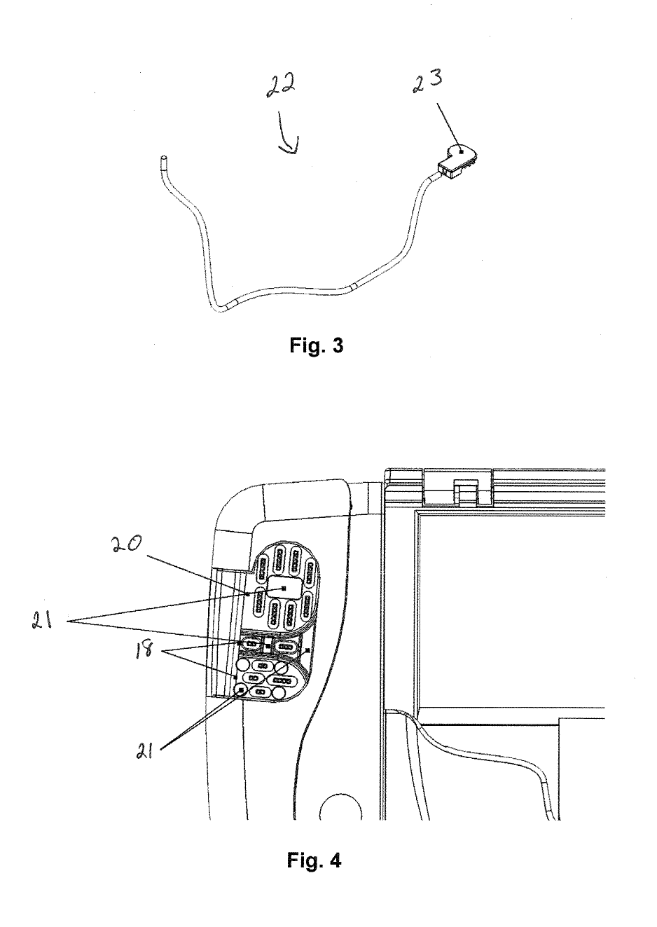

The device 10 may be adapted to connect with one or more wires used for performing a medical test, which may include electrophysiological test wires 22. For example, the left outrigger 16 may comprise various peripheral equipment ports 18 for the interaction between the device 10 and numerous proprietary peripheral equipment, as well as a connector port 20 that allows for the interaction between the device 10 and the electrophysiological test wires 22. The output signals from these wires may be delivered through circuitry in the left outrigger 16, and may be processed for display on a mobile computing device 26 (such as by direct connection 36, via wires, or wirelessly). As shown in FIG. 3, the electrophysiological test wire 22 may include a head connector 23 for connecting to the connector port 20. FIG. 4 illustrates a more detailed view of the peripheral equipment ports 18 as well as the connector port 20.

Any of these ports 18,20 may include one or more magnets 21 for maintaining contact between the test wires 22 and the port or ports 18, 20. As can be seen in the illustrated embodiment, any of the ports 18, 20 may include an asymmetrical configuration, at least along one axis, such that proper connection for communication between a head connector 23 and a port 18, 20 may occur only under a single orientation of the head connector.

With further reference to FIG. 1, the centralized shell 12 is adapted to house the mobile computing device 26, which may be in the form of a tablet or laptop computer. The mobile computing device 26, may serve to display one or more aspects of the medical test being performed, such as in a graphical format. The device will normally be present at the site of the testing and connected directly to the subject, but it should be appreciated that a professional for observing and interpreting the signals being displayed may be located at a remote location. To facilitate the review by this person, the signals may be transmitted remotely to the location of the professional, such as by a wireless transmission.

The centralized shell 12 has a slot that allows the mobile computing device 26 to slide in and fit securely within the enclosure. In addition, the centralized shell 12 includes a window 28 which allows access to the keyboard and track pad of the mobile computing device 26 in order for the user to have full access to the mobile computing device 26 while secured in the centralized shell 12. On the topside of the right outrigger 14 is a cut out 30 that allows access to a plunge button 32 that may actuate the power on/off switch of the mobile computing device 26 to be controlled while the mobile computing device 26 is securely locked inside the centralized shell 12.

As can be seen in FIG. 2, on a rear side of the right outrigger 14, various auxiliary ports 34 may be provided, such as USB and/or HDMI ports. The electronics located inside the left outrigger 16 may gain power from and communicate data over a direct connection 36 to the mobile computing device 26. This direct connection 36 consists of, but is not limited to, USB, wireless or Bluetooth communication.

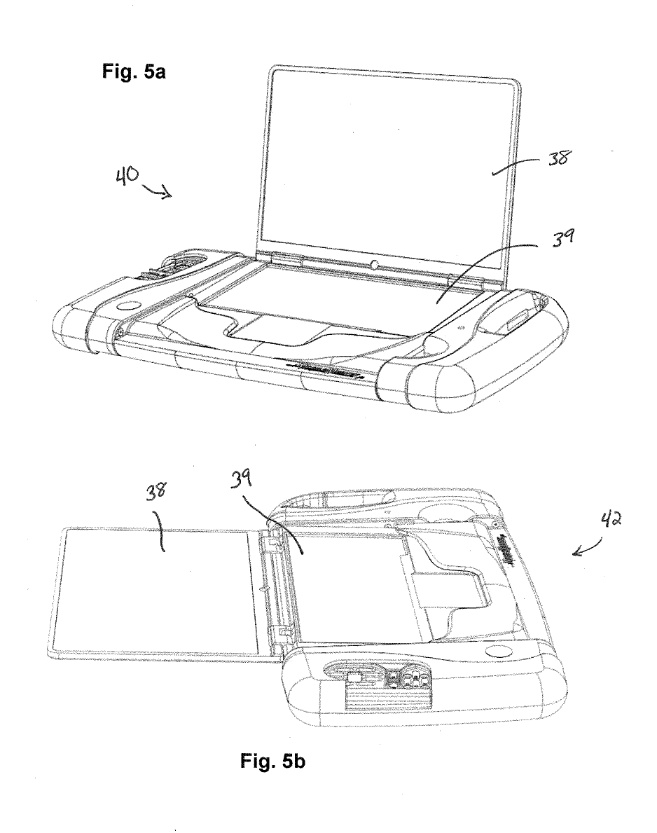

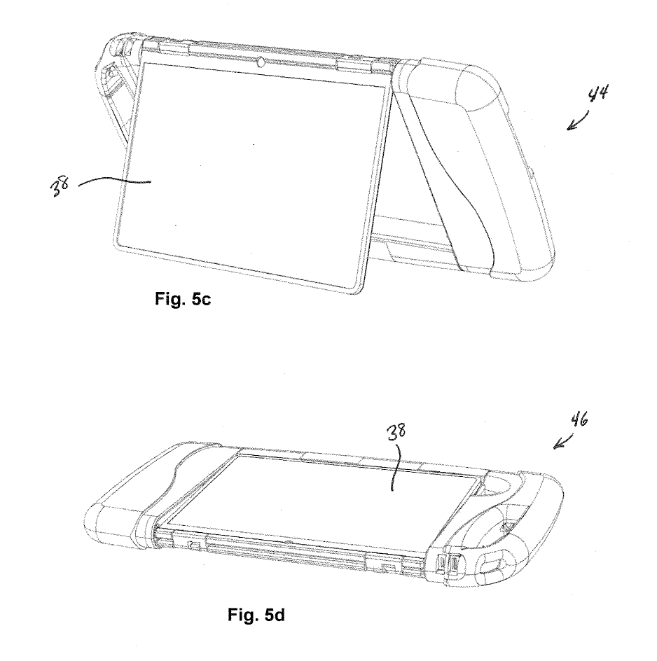

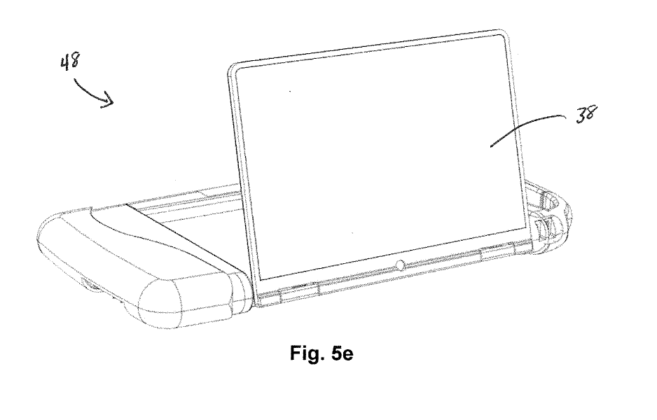

With reference to FIGS. 5a-5e, the device 10 is shown in a number of orientations that allow for flexibility of placement while in use. As shown in FIG. 5a, a keyboard input mode 40 allows the device 10 to operate in a similar orientation to a laptop for data input using the keyboard and track pad, such as with the screen 38 facing forward over the keyboard 39 facing upward when the device 10 is in an upright position. FIG. 5b illustrates a lateral input mode 42 allows the device 10 to operate while laying flat. In this orientation, both the screen 38 and the keyboard 39 face generally the same direction, namely upward when the device 10 is in an upright position. Lateral input mode 42 allows for either interaction using the touch screen interface or keyboard/track pad. With reference to FIG. 5c, a touch screen A-frame mode 44 is illustrated. This configuration allows the device 10 to be positioned in an A-frame orientation, with the screen 38 facing a generally opposite direction from the keyboard 39. Operation in the touch screen A-frame mode allows for use of a touch screen interface, such as screen 38, while the device 10 propped up for upright viewing. A tablet mode 46 is illustrated in FIG. 5d, which allows the device 10 to fold such that the keyboard 39 and track pad are rotated to face down, while the screen 38 is rotated to face up. This provides the user with the usability and convenience of a tablet while maintaining a small footprint. FIG. 5e illustrates a display mode 48, in which the screen 38 is positioned generally upright, while maintaining the keyboard 39 and track pad facing generally downward. The display mode 48 may also incorporate a screen support (not pictured) for maximum stability of the touch screen while in use. When not in use the screen support may recess into the base of the centralized shell 12.

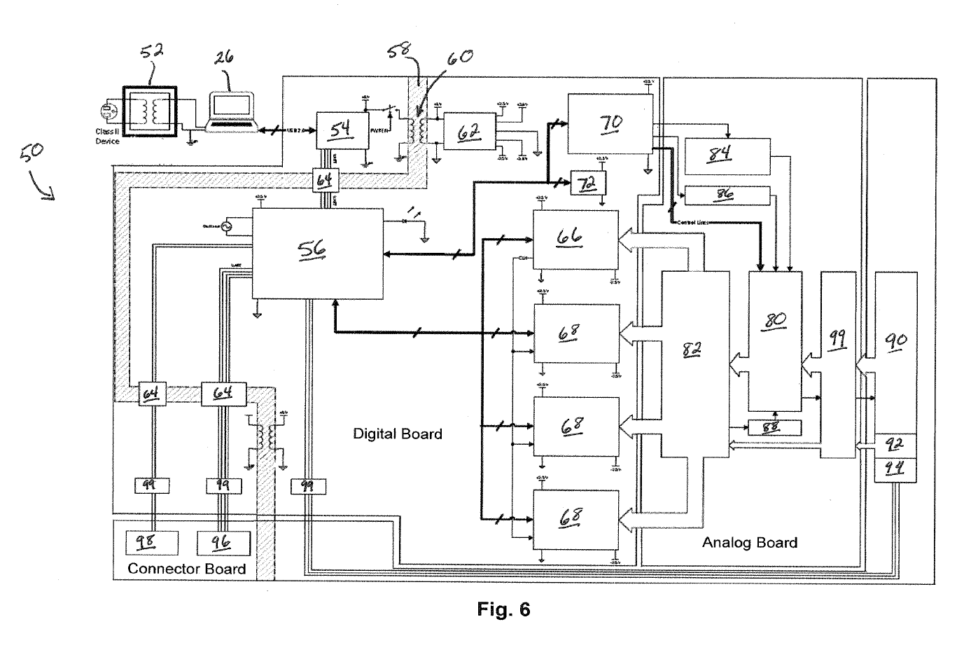

FIG. 6 illustrates a system 50 of the analog, digital, and connector components housed in left outrigger 16. In one aspect, the system 50 may be considered a CLASS II system, meaning that it has no protective earth ground connection. A power supply 52 may be provided for powering the system, such as a 120 V AC, 45 W power supply. The power supply 52 may be double insulated, providing two times Means of Operator Protection (MOOP).

The system 50 may be plugged in and powered via a 110 V outlet, or may be unplugged and run on battery power from the mobile computing device 26. The power supply 52 may output a voltage (e.g. 20V DC) to the mobile computing device 26. In one aspect, the mobile computing device may be a Lenovo Thinkpad, and may run a software program, such as ElectroTek Control Software, wherein the software program may be adapted to store, display, and transmit data collected from the system 50. Communication between the mobile computing device 26 and the system 50 may be accomplished via a port, such as a USB 2.0 port (500 mA max). The custom hardware of the system 50 may be powered via the USB 2.0 port. In addition, communication between the system 50 and the software on the mobile computing device 26 may be accomplished over the USB 2.0 port.

In one embodiment, the system 50 may include a plurality of circuit boards, which may include a digital board, an analog board, and a connector board. The system 50 may include a USB 2.0 bridge 54, which may be adapted to function as a translator between the mobile computing device 26 and a master embedded controller 56 of the system. The USB 2.0 bridge 54 may run the entire USB 2.0 protocol stack and may contain an internal data buffer. This bridge 54 may further allow USB communication (including overhead) to occur in parallel with other activities of the master embedded controller 56. In addition, the bridge 54 may be adapted to power down the system 50 in the event that the mobile computing device 26 issues a USB Suspend command, which has special low power draw requirements via the PWREN signal.

An isolation barrier may be provided in the system as a means of providing Means of Patient Protection (MOPP). The isolation barrier may include a plurality of elements, and may include one or more of air gaps and creepage distances, solid insulation, and isolation hardware that may provide two times MOPP. In one aspect, the isolation barrier may include a transformer 60, such as a 1:1 isolation transformer, for powering the system 50. During normal operation (i.e. without the USB Suspend command), the bridge 54 may allow USB voltage (e.g. +5V DC) to flow through a transformer 60.

A power management block 62 may be provided for transforming, conditioning, regulating, and protecting all power for the rest of the system 50. This power is supplied through the transformer 60, and may convert USB bus voltage to the various voltages needed for the system. In one aspect, the power management block may include ferrite power filters, and various protection features, which may include fuses and/or over-current protection.

In another aspect of the isolation barrier 58, the bridge 54 may translate communication (such as USB communication) to an embedded system protocol, such as UART, which flows across the isolation barrier through an isolation driver 64. This protocol may allow for communication across the isolation barrier, which may take the form of magnetic, capacitive, optical coupling technology, or any other technology for communicating across the isolation barrier.

With further reference to the master embedded controller 56, this element may function as the “brain” or controller for the overall system 50. The master embedded controller 56 may comprise a microcontroller, a microprocessor, DSP, FPGA, CPLD or other programmable logic, a custom ASIC, or similar embedded device and associated hardware (which may include an oscillator and program memory. The master embedded controller 56 may be adapted to manage and/or coordinate all activities of the system, including data collection, synchronization, peripheral device communication, event marker detection, etc. In another aspect, the master embedded controller 56 may configure the analog to digital converters 66, 68. In a further aspect, the master embedded controller 56 may be adapted to manage and configure a slave embedded controller 70, which may in turn control secondary tasks such as controlling a multiplexor (MUX) circuit 80. Additionally, the master embedded controller 56 may encapsulate and/or transmit collected data to the computing device 26 via the USB bridge 54 using a custom protocol.

The system 50 may further include a non-volatile memory element 72, which may provide additional memory to be used by the master embedded controller 56 for configuration, data buffering, etc. This memory element 72 is non-volatile, such that it will remember data even when power is not supplied. The non-volatile memory element 72 may communicate with the master embedded controller 56 via an SPI bus.

With further reference to the slave embedded controller 70, this slave controller may comprise a microcontroller, a microprocessor, DSP, FPGA, CPLD, or other programmable logic, a custom ASIC, or similar embedded device and associated hardware, such as an oscillator or program memory. This slave embedded controller 70 may be adapted for addressing delegated activities, such as impedance circuit management, calibration circuit management, and MUX circuit management. The slave embedded controller 70 may communicate with the master embedded controller 56 via an SPI bus. In a further aspect of the present invention, the system 50 may include the USB bridge, the main controller, and slave controller, and or various combinations thereof into a single device.

In addition, the system 50 may include one or more analog to digital converters (ADCs) 66, 68. These analog to digital converters may be adapted to simultaneously sample data for a plurality of channels (e.g. up to 32 channels) at 24 but resolution. The ADCs 66, 68 may be capable of interfacing to both differential and single ended signals. In one aspect, the ADCs 66, 68 may convert analog signals into a digital form that can be read by the main embedded controller 56. In the case of a plurality of ADCs, there may be one master ADC 66, and one or more slave ADCs 68. The master ADC 66 may transmit a synchronization signal to the slave converters to ensure simultaneous sampling. One or more of the ADCs 66, 68 may communication to the main embedded controller 56 via SPI bus. In a further aspect the ADCs may include programmable gain amplifiers that can be configured by the main embedded controller 56 to amplify the signal with various gains. In another aspect, the ADCs may be capable of sampling at various data rates, as configured by the main controller.

In one embodiment, the bridge 54, the master embedded controller 56, the transistor 60, and power management block 62, the ADCs 66, 68, the slave embedded controller 70, and the non-volatile memory element 72 may be included on the digital board.

The system 50 may further include a MUX 80 and/or analog switches. The MUX 80 may comprise a programmable circuit capable of switching the calibration and impedance circuits into and out of various signal paths to test various combinations of sensors and amplifier circuits. In addition, an analog signal conditioning element 82 may be provided. This analog signal conditioning element 82 may comprise stages of analog circuitry adapted to condition bio-physiological signals for sampling by the ADCs 66, 68. The analog signal conditioning element 82 may include one or more buffers, amplifiers, low pass filters, high pass filters, or any other elements capable of manipulating analog signals.

An impedance measurement circuit 84 may be provided as well. This impedance measurement circuit 84 may comprise a voltage controlled current source (VCCS) that may inject a small amount of current into the body of a patient and into circuitry for the interpretation of the electrode/skin impedance of various sensor connections. The impedance measurement circuit 84 may utilize the MUX 80 to switch between sensor combinations.

In addition, a calibration circuit 86 may be provided for calibration purposes. Specifically, the calibration circuit 86 may be adapted to inject a small square wave signal into the front of the analog signal conditioning element 82 via the MUX 80 for the purpose of calibrating and testing all data acquisition hardware. The calibration circuit 86 may utilize the MUX 80 to switch between various analog circuits.

In another aspect of the system 50, an active neutral element 88 may be provided for inversion of a reference signal characteristic of the common mode voltage on the body that is output back to the body with a current limited output that increases the common mode rejection of the system by decreasing common mode noise.

In one embodiment, the MUX 80, the analog signal conditioning element 82, the impedance measurement circuit 84, the calibration circuit 86, and the active neutral element 88 may be provided on the analog board.

In a further aspect, the system 50 may include a patient connection 90, which may comprise a single connection which includes receivers for all applied part signals. One or more differential sensor inputs 92 (e.g. floating sensors) may also be provided in order to accommodate an expanded electrode set, such as additional EEG or EMG channels for a larger data set or artifact removal. Additionally, a cable code 94 may be provided, which may be adapted to identify the type of patient cable that is plugged into the system.

In addition, one or more peripheral connections 96 may be provided for communication between the master embedded controller 56 and one or more peripheral devices , which may include a photic stimulator and/or a handheld nerve stimulator (not pictured). In one aspect, the peripheral connection 96 may be magnetic in nature. An isolation driver 64, such as a UART interface, may be provided to allow for communication across the isolation barrier 58 between the master embedded controller 56 and the peripheral connection 96, such as via one or more control lines. In another aspect, the peripheral connection 96 may not power the peripheral device(s). In this aspect, the power isolation is only used for powering the isolated side of the isolated line driver.

In a further aspect of the system 50, an event marker connection 98 is provided. The event marker connection 98 may comprise a magnetic connection that interfaces to a hand held pendant switch (not shown) used as an event marker. The event marker may be a single pole single throw (SPST) switch that may also have an LED, powered from the isolation transformer. The event marker may be utilized to mark a specific point in time where an event may have occurred. Events may include seizures, artifact / noise or physiologic abnormalities.

In one embodiment, the patient connection 90, the differential sensor inputs 92, the cable code 94, the peripheral connections 96, and the event marker connection 98 may be provided on the connector board.

At various positions throughout the system 50, one or more electrostatic discharge (ESD) protection elements 99 may be provided to protect against ESD discharges, in order to prevent damage to internal circuitry, such as per IEC 60601 regulations.

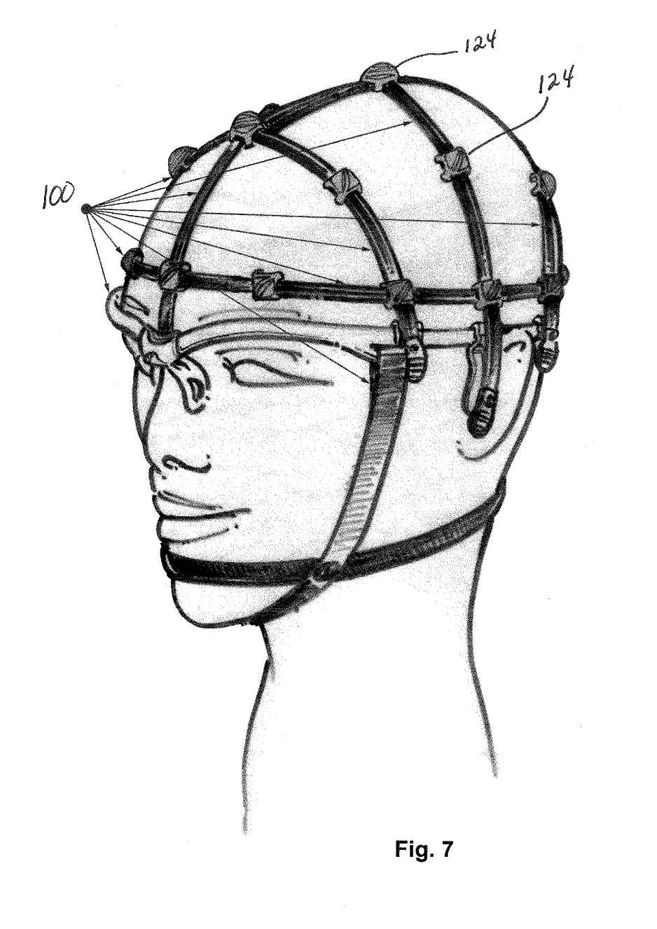

As can be seen in FIGS. 7-10b, in another embodiment of the present invention, a head apparatus 100 is disclosed. This head apparatus 100 may be used in selectively locating one or more electrophysiological signal gathering devices with respect to a patient's head. The electrophysiological signal gathering devices may comprise electrodes, wires, leads, or any other instrument capable of gathering said electrophysiological signals from the patient. Placement of the apparatus 100 and the electrodes, wires, leads, etc. on the patient may allow a clinician to collect and measure the various electrophysiological signals from the patient, such as in the conduction of an electroencephalograph (EEG).

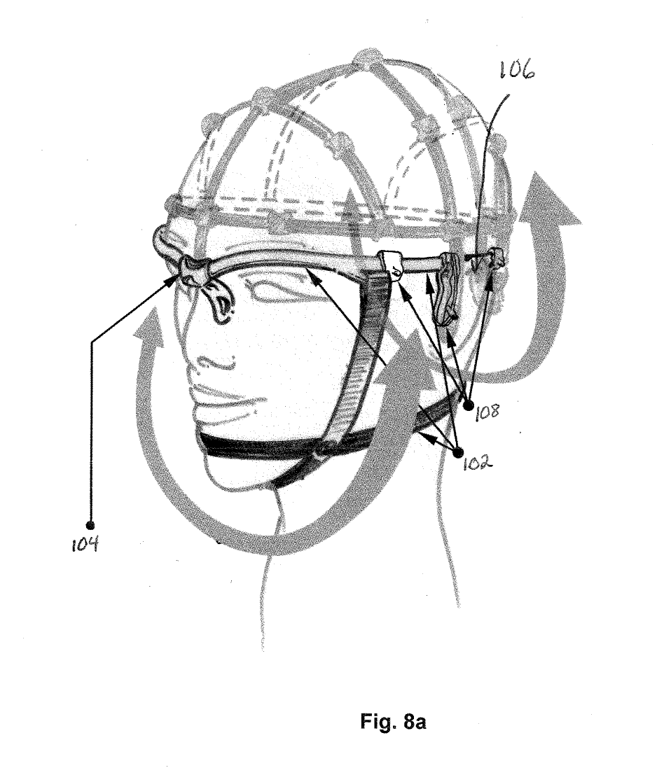

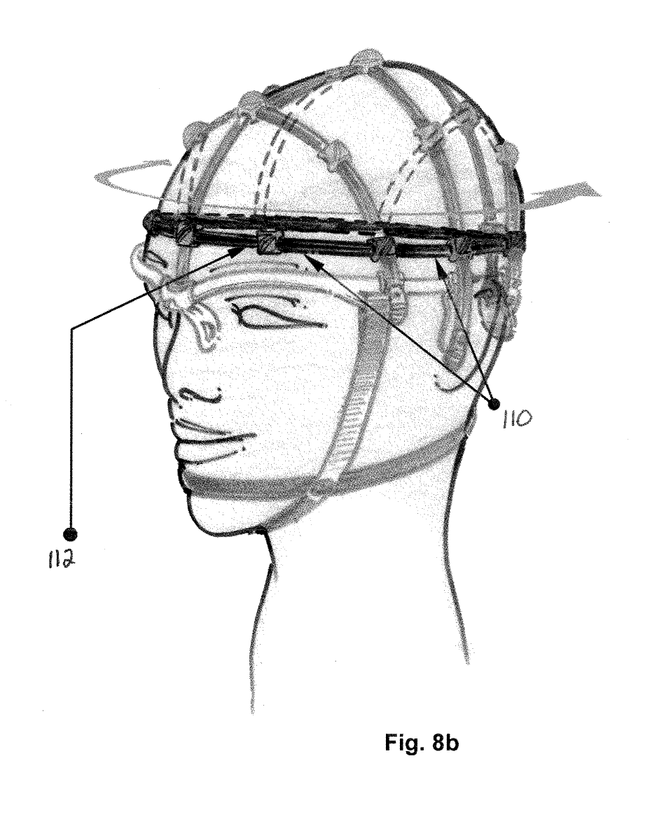

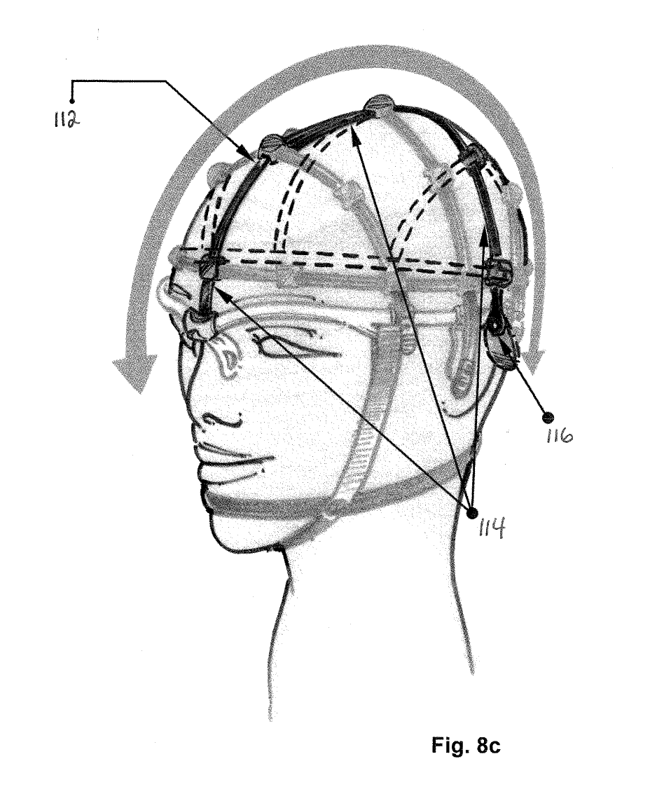

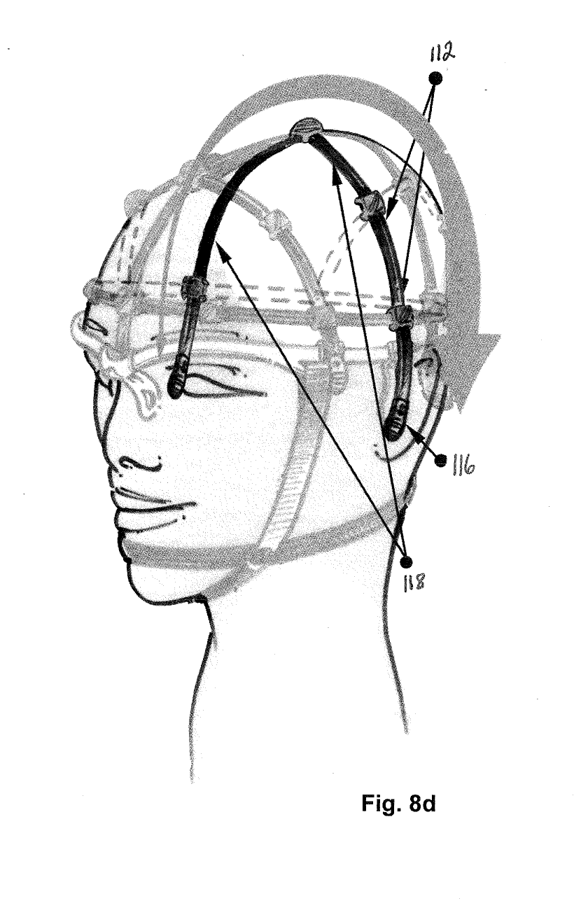

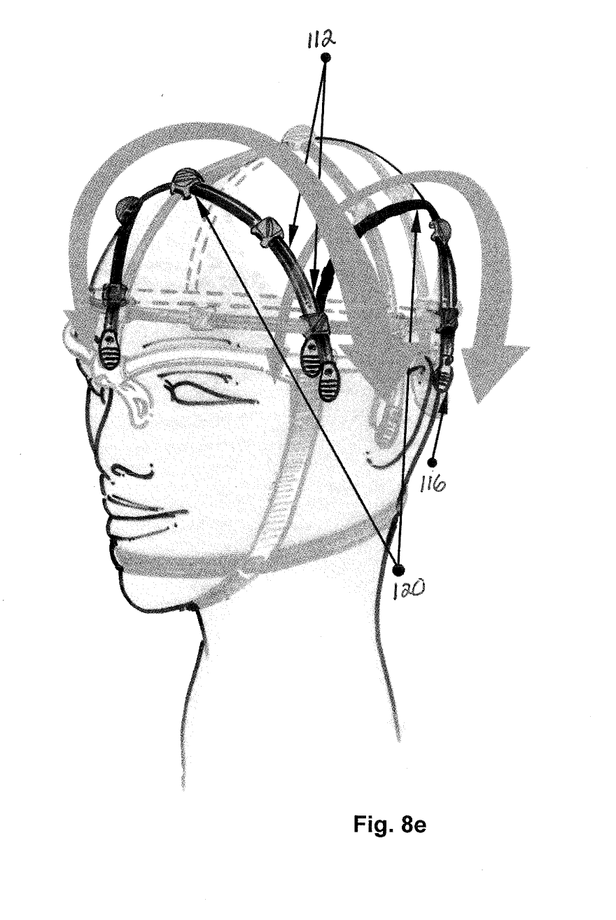

In the illustrated embodiment, FIG. 7, the head apparatus 100 is shown in its full form. The head apparatus 100 comprise a combination of individual full bands which may be attached to one another, a single unit with all appendages permanently attached to one another, or some combination of these two scenarios. FIGS. 8a-10b illustrate the details of the components of the head apparatus 100 and outline various characteristics thereof.

FIG. 8a illustrates a primary alignment band 102. The primary alignment band 102 may comprises a first piece for generally surrounding a circumference of the patient's head, and may further include one or more straps for securing the head apparatus 100 with respect to the patient's chin. The primary alignment band 102 may include materials such as, but not limited to, thin gauge metal, nylon, pliable plastic, rubber or vinyl.

In one aspect, the primary alignment band 102 may include three main functional elements. The first is the nose support and Nasion (Nz) location module 104. This module may rest on and/or be formed around the bridge of the nose and may include of a soft, pliable element comprising a material such as, but not limited to, silicon, gel or foam. The nose support and Nasion (Nz) location module 104 may include a grommet insert on a backside (i.e. towards the bridge of the nose when worn) that allows for attachment of an alignment/support band or apparatus. The Nasion (Nz) is located between the eyes on the bridge of the nose. The nose support may establish the center point for the Nasion (Nz) location and allow an anchor point for the alignment band, which then helps guide the remainder of the band placements.

A second functional element of the primary alignment band 102 is the tension module and Inion (Iz) location device 106. This device allows the primary alignment band 102 to increase or decrease its circumference without removing the band from the head. In one aspect, the tension module and Inion (Iz) location device 106 may comprise a sliding adjustable clip with external characteristics including a plastic, rubber, foam or metal housing. For example, the sliding adjustable clip may include a dial crank for adjusting the relative position of each of two side elements associated with the tension module and Inion location device. In another aspect, the tension module and Inion (Iz) location device 106 may have a grommet insert, such as on the backside (i.e. towards the head when worn), that allows for the attachment of an alignment/support strap or apparatus. Once in place, the nose support and Nasion (Nz) location module 104 and the tension module and Inion (Iz) location device 106 may establish the primary locations (Nz & Iz) for an electroencephalograph (EEG) utilizing industry standard electrode placement systems.

A third functional element of the primary alignment band 102 is the presence of one or more sliding anchors 108, which may be located along the primary alignment band 102. These sliding anchors 108 allow for the attachment of alignment/support bands or apparatuses to the primary alignment band 102.

As illustrated in FIG. 8b, the head apparatus 100 may include a main horizontal band 110. This main horizontal band 110 may be adapted to wrap circumferentially around the head and may comprise a pliable material with linear proportionate stretching capabilities such as, but not limited to, silicon, elastic or polypropylene. This linear proportionate stretching capability allows a band or strap to stretch equally along an axis thereof, such that a ratio between at least two distances between at least two pairs of locations along the band or strap remains constant as the band or strap stretches. The main horizontal band 110 may include markings 112 for accurate measurement of the head. These markings may be embedded in the main horizontal band 110 and may reflect relative distances from a given location on the band 110. For example, the markings may comprise a ruler for measuring distance along the band. These markings may follow appropriate electrode spacing requirements for the various standard electrode spacing systems such as, but not limited to, the International 10/20 system.

The head apparatus 100 may further include a central vertical band 114, as shown in FIG. 8c. The central vertical band 114 is adapted to run from a location on the front of the patient's head to a location on the rear of the patient's head. For example, the central vertical band 114 may run from the nose support & Nasion (Nz) location module to the tension module & Inion (Iz) location device 106. In one aspect, at each end of the central vertical band 114 a pull tab 116 may be provided for connecting directly to grommets located in the nose support and Nasion (Nz) location module 104 and the tension module and Inion (Iz) location device 106. This central vertical band 114 may comprise a pliable material with linear/proportionate stretching capabilities such as, but not limited to, silicon, elastic or polypropylene. The central vertical strap 114 may include markings 112 for accurate measurement of the head, and said markings 112 may be embedded therein. These markings may follow appropriate electrode spacing requirements for various standard electrode spacing systems such as, but not limited to, the International 10/20 system.

FIG. 8d illustrates a further aspect of the head apparatus 100, namely a central lateral band 118. The central lateral band 118 may be adapted to fit laterally over a central portion of the patient's head, such as from one ear to the other. At each end of the central lateral band 118, one or more pull tabs 116 may be provided for connecting directly to the sliding anchors 108 located along the primary alignment band 102. This central lateral band 118 may comprise a pliable material with linear/proportionate stretching capabilities such as, but not limited to, silicon, elastic or polypropylene. The central lateral strap 118 may include markings 112 for accurate measurement of the head. These markings may follow appropriate electrode spacing requirements for the various standard electrode spacing systems such as, but not limited to, the International 10/20 system.

As shown in FIG. 8e, front and rear lateral bands 120 may be included in the head apparatus 100. The front and rear lateral bands 120 are adapted to fit laterally over the patient's head along a front portion thereof and along a back portion thereof, respectively. For example, the front lateral band may be adapted to fit laterally over the patient's head anterior to the ears, while the rear lateral band may be adapted to fit laterally over the patient's head posterior to the ears. At each end of the front and rear lateral bands 120 may be a pull tab 116 that allows the bands to attach to the sliding anchors 108 located on the primary alignment band 102. These front and rear lateral bands 120 may comprise a pliable material with linear/proportionate stretching capabilities such as, but not limited to, silicon, elastic or polypropylene. The front and rear lateral bands 120 may have markings 112 for accurate measurement of the head. These markings may be embedded in the bands and may follow the appropriate electrode spacing requirements for the various standard electrode spacing systems such as, but not limited to, the International 10/20 system.

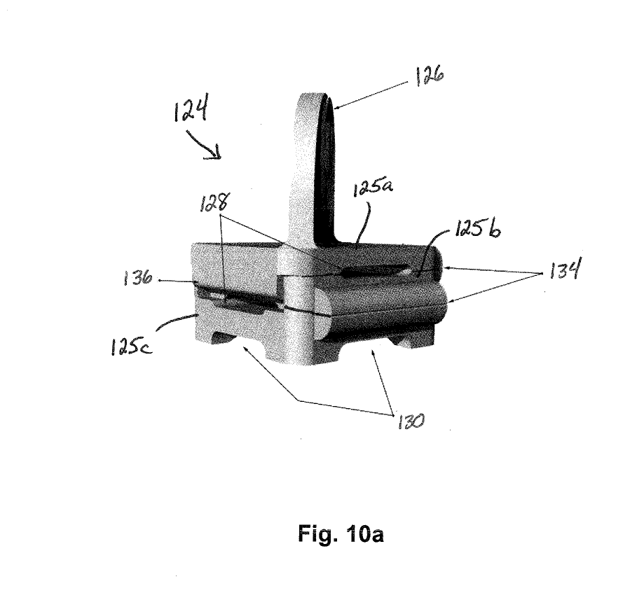

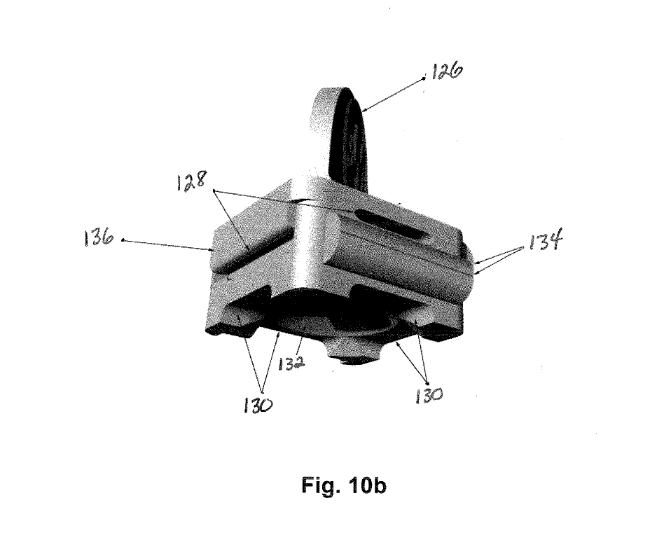

Any of the above bands and straps may intersect with one another and may be connected at their intersections by utilizing a connector 124 as described in further detail below and in FIGS. 10a and 10b.

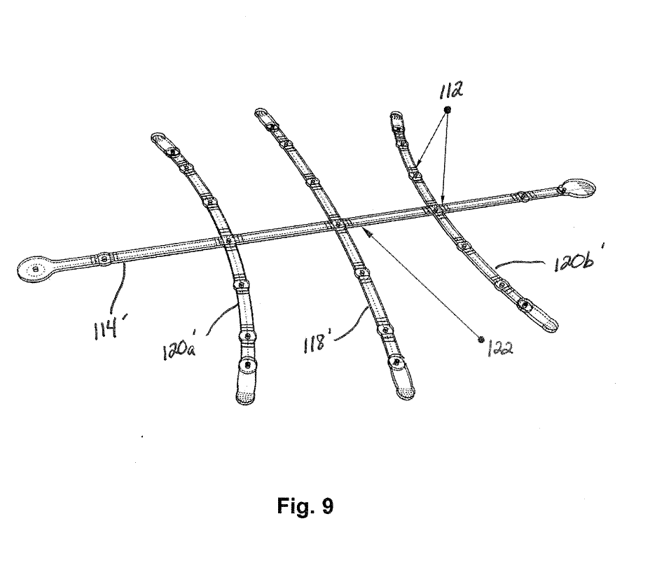

In an alternate embodiment, as illustrated in FIG. 9, a central and lateral band system 122 may be provided. This central and lateral band system 122 may comprise a central vertical band 114′, a central lateral band 118′, and front and rear lateral bands 120a′, 120b′, said bands forming an integrated, self-contained unit that does not require the use of connectors to hold the bands together. In one aspect, the central and lateral band system 122 may be adapted to attach to the patient's head as a single unit. At the ends of the vertical and lateral bands 114′, 118′, 120a′, and 120b′ of the central and lateral band system 122, one or more pull tabs 116 may be provided for directly connecting the central and lateral band system 122 to the sliding anchors 108 located along the primary alignment band 102. One or more connectors 124 may be provided along the central and lateral band system 122 that allow for the placement of various electrophysiological signal measurement units, such as, but not limited to, electrodes, wires and leads.

The central and lateral band system 122 may comprise a pliable material with linear/proportionate stretching capabilities such as, but not limited to, silicon, elastic or polypropylene. The central and lateral band system 122 may include markings 112 for accurate measurement of the head, and said markings may be embedded in the bands. These markings may follow appropriate electrode spacing requirements for various standard electrode spacing systems such as, but not limited to, the International 10/20 system.

Figures 10a and 10b illustrate one embodiment of a connector 124 that may be used in placing any electrophysiological signal gathering devices such as, but not limited to, electrodes, wires or leads, as well as serving as a connection device to the various bands that are outlined in FIGS. 8a-9. The connector 124 may include an exterior material of plastic or rubber. As illustrated in FIG. 10a, the connector 124 may include first, second, and third layers 125a, 125b, 125c, which may be articulatable with respect to one another.

In addition, a vertical tab 126 may be provided on top of the connector 124. This vertical tab 126 may serve as a way to handle the connector and reposition it on a patient if necessary. The vertical tab 126 also serves as a way to close the connector on itself, such as by providing a point of leverage for articulating a hinge 134, as outlined below.

In addition, one or more horizontal slots 128 may be located on the sides of the connector 124. The horizontal slots 128 may be formed by articulating the three layers 125a, 125b, 125c, of the connector 124 to close on themselves. The various bands associated with the head apparatus 100 can pass through the horizontal slots 128, which allow the connector 124 to slide along the straps in order to get proper placement of the electrophysiological wires.

In another aspect of the connector 124, one or more receivers such as wire slots 130 may be located along the bottom of the connector 124. The wire slots 130 allow for various electrophysiological wires to move in and out of the connector 124, while the connector maintains a flat connection with the surface of the head. The wire slots 130 allow for an electrophysiological wire, which may be glued to the head, to slip in and out of the wire slot 130 without having to remove the straps or bands. In this way, the configuration and placement locations of the headgear may remain constant even in the event of wires being added or taken away.

In a further aspect of the connector 124 as illustrated in FIGS. 10a and 10b, at least one hinge 134 may be provided. As illustrated, two hinges 134 are present, a first hinge for allowing articulation between the first and second layers 125a, 125b of the connector 124, and a second hinge for allowing articulation between the second and third layers 125b, 125c of the connector 124. In an open position, the first hinge 134 allows for separation of the first and second layers 125a, 125b, such as for placement of a band or strap of the head apparatus 100 therein. In a closed position, the first hinge 124 may place the first and second layers 125a, 125b in close proximity or in contact with one another, thereby forming a horizontal slot therebetween for retaining the band or strap. In one aspect, the horizontal slot 128 may be sized to retain the band or strap therein, but to allow for a longitudinal sliding movement of the band or strap within the slot. This may assist in positioning the connector 124 in the appropriate position, as the connector may remain attached to the band or strap, but may be allowed to slide along said band or strap to the appropriate position. This appropriate position may be determined by the one or more markings 112 on the band or strap. Similarly, the second hinge 124 allows for similar open and closed positions with respect to the second and third layers 125b, 125c, for placement of another band or strap of the head apparatus, if necessary.

The connector 124 may also be provided with one or more snap connections 136. The snap connection 136 may assist in securing two of the three layers 125a, 125b, 125c, with respect to one another. In the illustrated embodiment, the snap connection 136 may include a folding flap associated with a first layer 125a, which may at least partially overlap a portion of the second layer 125b, thereby securing the first layer 125a to the second layer 125b. Similarly, a second snap connection 136 may be provided for securing the second layer 125b to the third layer 125c.

As can be more clearly seen in FIG. 10b, a recess 132 may be located along the bottom of the connector 124. This recess 132 may allow for the electrophysiological signal measurement unit, such as an electrode, wire, or lead, to be placed under the connector 124, while still maintaining a flat connection to the surface of the head.

The connector 124 may additionally include a controller and/or other internal electronics adapted for the acquisition and amplification of electrophysiological signals from the body. Additionally, the connector 124 may include electronics to support a wireless transfer of the amplified data to the mobile computing device 26.

In a further embodiment of the present invention, a process is disclosed for the configuration of a system for performing an electrophysiological test on a patient. This process may be adapted to configure a series of electrodes, such as those associated with a wiring harness for use in association with the device 10 of the present invention. For example, the system may be adapted to configure the electrodes associated with a head apparatus 100 of the present invention, for use with the portable device 10, in order to perform an electroencephalogram (EEG).

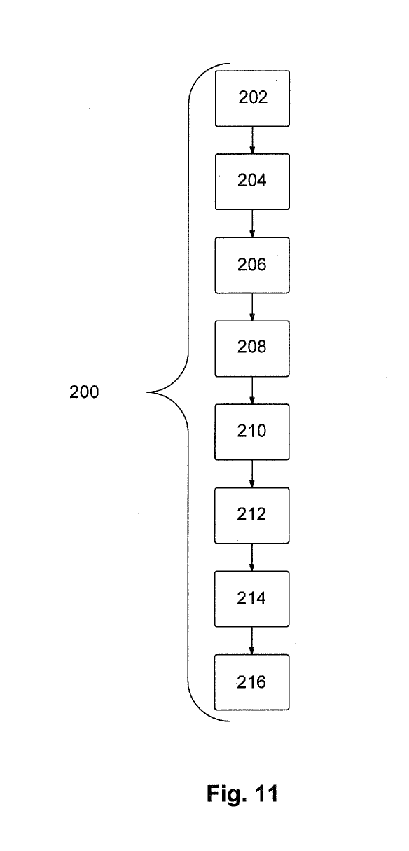

As illustrated in FIG. 11, a schematic of process of an electrode configuration module 200 is disclosed. This electrode configuration module 200 may be used to configure one of a plurality of different wiring harnesses associated with a plurality of different electrophysiological tests. The process may include a step of selecting and attaching an appropriate electrophysiological wiring harness associated with a desired electrophysiological test to be performed by the device 10. Each of a plurality of wiring harnesses adapted for a given test may be internally coded so that the software application associated with the device 10 may automatically recognize the wiring harness and the particular test to be run with said wiring harness. This automatic recognition of the wiring harness may occur through the use of a unique configuration of the wiring harness, such as the use of a unique resistor or series of resistors particular to said wiring harness. Through recognition of said unique configuration, the software may identify the particular test to be run.

The software may have access to an electrode placement diagram specific to the particular test to be run. In addition, said software may include a prompt to a user to begin configuration and/or placement of the wiring harness and/or the wires or electrodes associated therewith according to the electrode placement diagram. In one aspect, the electrodes associated with the wiring harness may be attached to the patient via the harness, as illustrated in step 202. For example, this may involve attaching the head apparatus 100 to a patient. The process 200 may include the step of accessing a configure electrodes module 204. This configure electrodes module 204 may prompt the user by signaling a given electrode on the electrode placement diagram, such as by flashing light associated with or representative of a given electrode, as noted in step 206. Upon receiving said prompt regarding a given electrode on the electrode placement diagram, the user may identify the corresponding electrode or electrode location on the wiring harness, as indicated at step 208. This may involve the user manually manipulating the electrode, such as by tapping, pressing and holding, or squeezing the corresponding electrode (or an actuator such as a button associated therewith) associated with the wiring harness. Upon the user identifying the corresponding electrode associated with the wiring harness, the configure electrodes module may auto-configure the identified electrode as corresponding to the electrode from the electrode placement diagram within the software system, as shown in step 210. This series of steps, including the prompting the user with an electrode on the electrode placement diagram, identification by the user of a corresponding electrode on the wiring harness, and auto-configuration of said identified electrode on the wiring harness may be repeated until all electrodes associated with the wiring harness have been auto-configured, as indicated at step 212.

An impedance check of the electrodes associated with the wiring harness may be completed, as indicated at step 214. This may be completed after all electrodes have been auto-configured within the system, or may be completed individually as a given electrode is auto-configured. This impedance check step 214 may lead to an indication step 216, that one or all of the electrodes have been configured and the software application is properly receiving data signals from the electrodes associated with the wiring harness. In one aspect, this indication step 216 may be in the form of a visual or audible cue.

The foregoing descriptions of several embodiments made according to the disclosure of certain inventive principles herein are presented for purposes of illustration and description. The embodiments described are not intended to be exhaustive or to limit the invention to the precise form disclosed and, in fact, any combination of the components of the disclosed embodiments is contemplated. Modifications or variations are possible in light of the above teachings. The embodiments described were chosen to provide the best illustration of the principles of the invention and its practical applications to thereby enable one of ordinary skill in the art to utilize the invention in various embodiments and with various modifications as are suited to the particular use contemplated. All such modifications and variations are within the scope of the invention when interpreted in accordance with the breadth to which it is fairly, legally, and equitably entitled.

1. An apparatus for use in association with a portable computing device in performing a plurality of different electrophysiological tests on a patient, said apparatus comprising:
a body including a receiver for connecting to and receiving at least a portion of the portable computing device; and
a controller for receiving a plurality of electrophysiological signals from the patient and for processing and transferring said electrophysiological signals to the portable computing device.

2. The apparatus of claim 1, wherein the body includes a central shell with at least one opening for allowing access to a keyboard or touch interface of the portable computing device when the portable computing device is received by and connected to said body.

3. The apparatus of claim 2, further including means for accessing at least one tactile button on the portable computing device.

4. The apparatus of claim 2, wherein the body further includes a first portion on a first side of the central shell and a second portion on a second side of the central shell, wherein the first portion includes the controller.

5. The apparatus of claim 4, wherein each of the first portion and the second portion includes a recessed grip adapted to allow a user to hold the body and the portable computing device.

6. The apparatus of claim 1, wherein the controller is adapted to be powered by the portable computing device.

7. The apparatus of claim 1, further including a connector port adapted to receive the plurality of electrophysiological signals from the patient via a plurality of different configurations of electrodes and further adapted to transmit said signals to the controller for processing.

8. The apparatus of claim 7, wherein the body further includes at least one port for direct electrical communication with an input port associated with the portable computing device.

9. An apparatus for use in locating and attaching electrophysiological signal transmitters to a head of a patient, said electrophysiological signal transmitters adapted to receive electrophysiological signals from the patient and transmit said signals to a system for processing said signals, said apparatus comprising:
a plurality of bands for positioning on the head of the patient, said bands adapted for linear proportionate elongation; and
a plurality of connectors for connecting the electrophysiological signal transmitters to the plurality of bands and for maintaining contact between the transmitters and the head of the patient.

10. The apparatus of claim 9, wherein at least one of the plurality of connectors is adapted to connect a first of the plurality of bands to a second of the plurality of bands.

11. The apparatus of claim 9, wherein at least one of the plurality of bands includes markings thereon for measuring a distance from a predetermined point.

12. The apparatus of claim 9, wherein the plurality of bands comprises a vertical band adapted for placement along a central portion of the head from front to back and a plurality of lateral bands adapted for placement over the head from side to side.

13. The apparatus of claim 12, wherein the plurality of bands further comprises a circumferential band adapted to substantially surround a circumference of the head.

14. The apparatus of claim 13, further including a primary positioning element for attachment to and location of the plurality of bands, said primary positioning element including a nose support and Nasion location element.

15. The apparatus of claim 14, wherein the primary positioning element further includes an Inion location element.

16. The apparatus of claim 12, wherein the vertical band and the plurality of lateral bands form an integrated, unitary element.

17. The apparatus of claim 9, wherein the connectors comprise a plurality of slots for slidably receiving at least one of the plurality of bands, and at least one receiver for receiving at least one of the electrophysiological signal transmitters.

18. The apparatus of claim 17, wherein the connectors further include at least two layers connected by a hinge, said hinge adapted for opening the at least two layers for placing at least one of the plurality of bands within at least one of the plurality of slots.

19. The apparatus of claim 17, wherein the connectors further include a controller for amplifying the electrophysiological signals and wirelessly transmitting said signals.

20. A method of configuring one of a plurality of wiring harnesses attached to a patient, said wiring harnesses including a plurality of electrodes for use in conducting one of a plurality of different electrophysiological tests on the patient, each of said wiring harnesses including an internal code specific to one of the plurality of different tests, said method comprising the steps of:
providing an electronic testing system adapted for connecting to each of the plurality of different wiring harnesses, said electronic testing system for receiving and processing electrophysiological signals from the patient;
identifying a first of the wiring harnesses connected to the electronic testing system by a first internal code associated with the first wiring harness;
identifying a fist map of a plurality of electrode locations associated with conducting a first electrophysiological test with the first wiring harness, said first map accessible by the electronic testing system;
prompting a user to identify each of the plurality of electrodes of the first wiring harness corresponding to the electrode locations;
receiving an input from the user identifying each of the plurality of electrodes of the first wiring harness; and
mapping each of the plurality of electrodes of the first wiring harness to each of the electrode locations of the first map.
